(19) United States
(12) Patent Application Publication
(10) Pub. No.: US 2023/0178202 A1
(43) Pub. Date: Jun. 8, 2023

(54) Methods and Systems for Managed Authorization Routing

(71) Applicant: CadenceRx, Tampa, FL (US)

(72) Inventor: Anthony Lentini, Waxhaw, NC (US)

(73) Assignee: CadenceRx, Tampa, FL (US)

(21) Appl. No.: 17/540,459

(22) Filed: Dec. 2, 2021

(51) Int. Cl. G16H 20/10

(52) U.S. Cl. CPC G16H 20/10

(57) ABSTRACT

Described herein is a system, method, and non-transitory computer-readable medium for managed authorization routing to identify, mitigate, and prevent potential issues that may occur when authorizing medications for patients. Also described herein is a system, method, and non-transitory computer-readable medium for generating a degrees of caution score utilized to approve or deny a medication authorization request.

### TECHNICAL FIELD OF THE DISCLOSURE

The present disclosure is directed to methods and systems for pharmaceutical analysis and patient claims management.

### BACKGROUND

There are shortcomings within the current healthcare system related to safe and effective medication management. It is not uncommon that patients receive medications that subsequently prove harmful to their health status as a result of caustic medication interactions, allergic reactions, over prescribing, and patient profile. Current health platforms often provide retrospective analysis after a medication has been dispensed and potential side effects or dangerous medication interactions have occurred. Therefore, there is a need for systems and methods for proactively ensuring that safe and cost-effective pharmaceutical decisions are made in order to improve the lives of patients by making prospective and concurrent clinical oversight a priority.

### SUMMARY

Some embodiments herein provide methods, systems, and non-transitory computer readable media for managed authorization routing to identify, mitigate, and prevent potential issues that may occur when authorizing medications for patients. Also described herein are methods, systems, and non-transitory computer readable media for generating a degrees of caution score utilized to approve or deny a medication authorization request.

According to one aspect, the described invention provides a system for managed authorization routing. The system includes a storage configured to hold a plurality of variables associated with a patient and a medication. Each variable of the plurality of variables are associated with a category of a plurality of categories. The storage is further configured to hold at least one threshold level to define at least one action to perform. The system further includes one or more processors in communication with the storage and configured to execute instructions, including instructions to receive a request to approve or disapprove a medication authorization request for the medication. The instructions further causing the computing system to score each variable of the plurality of variables. A score defines a risk associated with approving the medication. The instructions further causing the computing system to multiply each score assigned to each variable of the plurality of variables by a weight assigned to each variable to generate a weighted score for each variable. The instructions further causing the computing system to sum the weighted scores within each category of the plurality of categories to generate a score for each category. The instructions further causing the computing system to sum the scores for the plurality of categories to obtain a degrees of caution score. The instructions further causing the computing system to determine, based on the degrees of caution score and the threshold level, whether to automatically approve or deny the medication authorization request or route the medication authorization request to a user (e.g. claim manager) to approve or deny the medication authorization request. The instructions further causing the computing system to automatically approve or deny the medication authorization request, or display the degrees of caution score and an option to approve or deny the medication authorization request to a user via a graphical user interface.

According to another aspect, the described invention provides a method for managed authorization routing. The method includes storing, within a storage, a plurality of variables associated with a patient and a medication. Each variable of the plurality of variables is associated with a category of a plurality of categories. The method also includes storing, within the storage, at least one threshold level to define at least one action to perform. The method also includes receiving, via one or more processors in communication with the storage, a request to approve or deny a medication authorization request for the medication. The method also includes scoring, via the one or more processors, each variable of the plurality of variables. A score defines a risk associated with approving the medication. The method also includes multiplying, via the one or more processors, each score assigned to each variable of the plurality of variables by a weight assigned to each variable to generate a weighted score for each variable. The method also includes summing, via the one or more processors, the weighted scores within each category of the plurality of categories to generate a score for each category. The method also includes summing, via the one or more processors, the scores for the plurality of categories to obtain a degrees of caution score. The method also includes determining, via the one or more processors, based on the degrees of caution score and the threshold level, whether to automatically approve or deny the medication authorization request or route the medication authorization request to a user to approve or deny the medication authorization request. The method also includes automatically approving or denying, via the one or more processors, the medication authorization request, or displaying the degrees of caution score and an option to approve or deny the medication authorization request to a user, such as a claims manager, via a graphical user interface.

According to another aspect, the described invention provides a non-transitory computer readable medium including instructions that when executed by one or more processors to cause a computing device or system to perform any of the methods recited or claimed herein.

### BRIEF DESCRIPTION OF THE DRAWINGS

To assist those of skill in the art in making and using the described system and associated methods, reference is made to the accompanying figures. The accompanying figures, which are incorporated in and constitute a part of this specification, illustrate one or more embodiments of the invention and, together with the description, help to explain the invention. Illustrative embodiments are shown by way of example in the accompanying drawings and should not be considered as limiting. In the figures:

FIG. 1 schematically depicts a network diagram of computing systems, devices, networks and databases that could be employed in accordance with an embodiment described herein.

FIG. 2 illustrates an exemplary graphical user interface (GUI) for customizing weights assigned to variables and viewing authorization projections based on the assigned weights in accordance with an embodiment described herein.

FIG. 3 illustrates an exemplary GUI for reviewing and approving or denying a medication authorization request in accordance with an embodiment described herein.

FIGS. 4A and 4B illustrate an exemplary degrees of caution score and categories with associated scores as shown on an GUI in accordance with an embodiment described herein.

FIG. 5 is a block diagram of an example computing device that can be used to perform one or more steps of the systems and methods provided by exemplary embodiments.

FIG. 6 is a flow chart of an example process that can be executed in accordance with an embodiment described herein.

FIG. 7 is a chart demonstrating a reduction in turn-around time for prior authorizations using degrees of caution scores.

### DETAILED DESCRIPTION OF THE INVENTION

Embodiments are discussed in more detail referring to the drawings that accompany the present application. In the accompanying drawings, like and/or corresponding elements are referred to by like reference numbers.

Throughout the specification and claims, terms may have nuanced meanings suggested or implied in context beyond an explicitly stated meaning. Likewise, the phrase “in some embodiments” as used herein does not necessarily refer to the same embodiments nor does it necessarily refer to different embodiments. It is intended, for example, that claimed subject matter include combinations of example embodiments in whole or in part.

In general, terminology may be understood at least in part from usage in context. For example, terms, such as “and”, “or”, or “and/or,” as used herein may include a variety of meanings that may depend at least in part upon the context in which such terms are used. Typically, “or” if used to associate a list, such as A, B, or C, is intended to mean A, B, and C, here used in the inclusive sense, as well as A, B, or C, here used in the exclusive sense. In addition, the term “one or more” as used herein, depending at least in part upon context, may be used to describe any feature, structure, or characteristic in a singular sense or may be used to describe combinations of features, structures or characteristics in a plural sense. Similarly, terms, such as “a,” “an,” or “the,” again, may be understood to convey a singular usage or to convey a plural usage, depending at least in part upon context. In addition, the term “based on” may be understood as not necessarily intended to convey an exclusive set of factors and may, instead, allow for existence of additional factors not necessarily expressly described, again, depending at least in part on context.

Embodiments described herein include systems, methods, and/or non-transitory computer-readable medium for pharmaceutical analysis and managed authorization routing to identify, mitigate, and prevent potential issues that may occur when authorizing medications for patients. In some embodiments, a degrees of caution score is generated and utilized prior to a medication being dispensed to a patient. In some embodiments, the degrees of caution score and a decision to approve or deny a medication authorization request is routed to an appropriate resource, such as a claims manager. In some embodiments, an automated decision to approve or deny a medication authorization request is made based on customizable thresholds and the degrees of caution score. Some embodiments result in critical medical decisions within the medication management process occurring in a more efficient manner and patients achieving an improved health result.

While the disclosed embodiments and examples are generally directed to workers’ compensation claims’ management focusing on pharmaceutical analysis, the described systems and methods have similar application benefits along the entire health care management spectrum including automobile and group health.

In some embodiments, a method or system employs a rules engine to generate a degrees of caution score before a medication authorization request is authorized within a claims’ management process. For example, the medication authorization request may be a request associated with authorization for a new prescription, authorization for a renewal or refill of a prescription, and/or a medication requiring a prior authorization or pre-authorization. In some embodiments, medications that require prior authorization are broken into 4 segments: requests for medications that are on-formulary but require an authorization for a quantity, day supply, or maximum dollar override (commonly called group edits), medications that require authorization that are on-formulary but require authorization, request for a medication from a special list of medications often dictated by the state but customizable by a client, and lastly medications that are not on the formulary.

The rules engine implements a prospective risk assessment associated with medication dispensing. In some embodiments, the rules engine performs an analysis to develop a degrees of caution score based on a number of factors that provide insight into whether a medication should be approved or denied for a patient prior to the medication being dispensed to the patient. In some embodiments, the rules engine uses a combination of variables or data points, medical literature, population data, and/or advanced analytics to develop a degrees of caution score. In some embodiments, the degrees of caution score ranges from 1 and 100, where the higher the degrees of caution score, the more risk for complications, dependency, or improper use associated with approving the medication.

In some embodiments, the rules engine uses a combination of variables broken into 5 major categories: a Patient Profile category that includes variables associated with a patient profile, a Therapy Patterns category that includes variables associated with therapy patterns, a Prescribing Patterns category that includes variables associated with prescribing patterns, a Medication Specifications category that includes variables associated with medication specifications, and a Prescriber & Pharmacy Quality category that includes variables associated with prescriber and pharmacy quality. In such an embodiment, each variable is associated with at least one of these major category.

The Patient Profile variables include items that are pertinent to the patient (for example, patient age, age of the claim, injury types, working status, etc.).

The Therapy Patterns variables incorporate the complete scope of medications that patient is and/or has taken. Examples are cross reactive medications, cumulative Morphine Equivalent Dose (MED), duplication of therapy, polypharmacy, etc.

The Prescribing Patterns variables measure behaviors of the prescribing doctor. For example, this is used to identify doctors who are over prescribing various Class II medications (i.e. opioids, benzodiazepines, sleep agents and muscle relaxers) and combinations of these.

Medication Specifications variables includes specifics of the requested medication. Examples include the medication class, quantity, day supply, compound, repackaged, topical, Dispense As Written (DAW), previous approval, and atypical workers’ compensation medications.

Prescriber & Pharmacy Quality variables measures the potential use of multiple doctors or pharmacies by the patient (commonly used to hide medications or duplicate prescription usage), quality of the doctor and pharmacy network affiliation (for example, catching compounding pharmacies, mail order facilities, or less common distribution facilities).

For example, in some embodiments, the rules engine utilizes one or more of the following variables to determine a degrees of caution score: patient profile metrics, clinical expertise, prescribing patterns of the physician and previous disposition, claim age, length of therapy, medication class, medication quantity, day supply, duplicate therapies, cross-reactive medications, multiple pharmacies, multiple prescribers, transaction source, physician quality scores, total and cumulative opioid dosage (MED), medication testing, medication monitoring, use of compounds, use of repackaged medications, or high cost/brand only preference. In some embodiments, the module uses a combination of up to 54 variables.

In some embodiments, these variables are obtained from various sources. For example, a user may provide patient demographics and patient profile data through an eligibility graphical user interface (GUI); medication data may be provided by a prescribing pharmacy; therapy patterns and prescribing patterns may be developed by looking at a fill history for a patient and across a book of business; and pharmacy and prescriber quality scores may be gathered by studies and/or provided by clients.

In some embodiments, the rules engine scores each variable on a scale from 0 to 4 that defines the risk associated with approving the medication. For example, a variable score of 0 indicated no risk, a variable score of 1 indicates low risk, a variable score of 2 indicates medium risk, a variable score of 3 indicates medium-high risk, and a variable score of 4 indicates high risk. In some embodiments, the variable scoring is based on evidence-based medicine, incorporating published clinical studies into the criteria, and focusing on outcomes based medical literature. Each variable has various rules and/or formulas or data applications that results in a unique score for the variable.

In some embodiments, clinical expertise is utilized to define customizable, state defined, and injury-specific formularies for scoring variables. Managing appropriate off-formulary prescriptions is often complex for a variety of reasons, for example, the continual introduction of new medications, complex multi-medication regimens, escalating use of opioids, varying degrees of injury severities, state formularies, expensive brand medication outliers, and the volume of brand medications moving to generics. The scoring process evaluates the National Council for Prescription Medication Programs (NCPDP) rejection type based on custom and state formulary rules as a component of the variable scores. Medications that are on formulary but require authorization are less risky to approve as they are considered to be in line with therapeutic guidelines. In certain states that adhere to an ODG formulary and maintain an ‘N-Medication’ list that require special handling, the rules engine reflects this in the scoring of variables and the routing and decision module described below routes these medication authorization requests to specialized teams or individuals.

In some embodiments, the scoring of the variables is performed manually by a user, such as a clinician, based on evidence-based medicine, incorporating published clinical studies into the criteria, and focusing on outcomes based medical literature.

In some embodiments, the rules engine scores at least some of the variables before receiving a medication authorization request. For example, the rules engine may score variables associated with a variety of medications before receiving a medication authorization request. In some embodiments, the rules engine scores at least some of the variables after receiving a medication authorization request. For example, the rules engine may score variables associated with a patient after receiving a medication authorization request from the patient.

In some embodiments, the rules engine multiplies each assigned variable score by a weighting associated with the variable to generate a weighted score for the variable. Each variable has a default weighting; however, a user can customize the weighting to increase or decrease the impact of the variable. For example, in some embodiments, a score for a variable is multiplied by a default weighting from 0 to 5 (0 is used to eliminate the variable from the total, 1 is used to not increase nor decrease the impact of the variable) to generate a weighted score for the variable. Each variable has a default weighting that is used to increase/decrease the impact of that variable on the category score and overall degrees of caution score. This weight can be overridden by a user and/or modified to 0 to exclude that variable.

Each of the variables can be included or excluded and/or weighted when calculating the degrees of caution score. For example, Morphine Equivalent Dose (MED level) is an example of a common variable that is weighted at 2 or 3. This is dependent on the user’s evaluation of the MED level as a metric for risk. If the user believes that their patient base is affected more by this metric, the user can increase the weighting. The same technique is often used in targeted medications, topicals, cross reactive, and high cost medications.

In some embodiments, after each variable is assigned a score and a weighted score is generated, the rules engine sums the weighted scores of the variables for each category (for example, Patient Profile, Therapy Patterns, Prescribing Patterns, Medication Specifications, and Prescriber & Pharmacy Quality), thereby generating a score for each category. The rules engine sums all category scores to obtain a degrees of caution score.

Below is an example algorithm for aggregating values used in the Patient Profile category. The algorithm includes scoring metrics used within the model, such as the assigned variable score, the weight, and the weighted score:

{
“CategoryId”: 1
“Category”: “Patient Profile”,
“SubCategory”: “Patient Age”,
“Weight”: 1.0,
“Score”: 2.0,
“WeightedScore”: 2.0
},
{
“CategoryId”: 2,
“Category”: “Patient Profile”,
“Subcategory”: “Claim Age”,
“Weight”: 1.0,
“Score”: 4.0,
“WeightedScore”: 4.0
},
{
“CategoryId”: 3,
“Category”: “Patient Profile”,
“SubCategory”: “Multiple Claims”,
“Weight”: 1.0,
“Score”: 0.0,
“WeightedScore”: 0.0
},

In some embodiments, the rules engine uses all available variables when generating a degrees of caution score. In some cases, whether the rules engine uses a particular variable when generating the degrees of caution score is dependent on an availability of data and a user’s determination to include or exclude the variable. In some cases, a variable isn’t applicable (for example, if the medication is not an opioid) or if the variable is not available for the patient. Variables may be dependent on what a user (such as a client) provides to manage a claim. In some cases, a variable may not be available because a client does not provide the variable, or there is not enough history for a patient to make a determination for a variable. While the majority of the variables are dependent on a medication, information on a patient, a pharmacy, and a prescriber are also taken into account when generating a degrees of caution score.

In some embodiments, variables are captured at run time from a combination of sources, including a JSON object, SQL transformations, and/or pulled from static sources.

In some embodiments, the degrees of caution score is generated during the process of creating a medication authorization request.

In some embodiments, the rules engine performs scoring on all medication authorization requests as a way to develop a robust dataset. This includes off-formulary medications and formulary medications.

Some embodiments described herein further include a system, method, and/or non-transitory computer-readable medium for managed authorization routing to ensure that the proper action is taken in response to a degrees of caution score and/or to provide the right level of information to the right person at the right time. In some embodiments, a method or system employs a routing and decision module that determines whether to automatically approve or disapprove medication authorization requests, or route medication authorization requests to users (for example, examiners or clinical groups) to approve or disapprove the medication authorization request. The determination is based on degrees of caution scores, customizable threshold levels, category weighting, user’s predefine responses, routing rules, and/or escalation procedures.

In some embodiments, the threshold levels are defined by a user to determine a particular action. In some embodiments, a user instructs the routing and decision module to take an action based on the degrees of caution score and threshold levels. For example, all medication authorization requests with degrees of caution scores below 20 are automatically approved, all medication authorization requests with degrees of caution scores between 21 and 69 are sent to an examiner for review, all medication authorization requests with degrees of caution scores between 70 and 89 are routed to the clinical review team for review, and all medication authorization requests with degrees of caution scores above 90 are automatically denied. This may create efficiency for examiners to focus on the medication authorization requests that require manual/human review.

In another non-limiting example, a client may utilize the degrees of caution score to route a medication authorization request of higher scores (i.e., above a degrees of caution score of 80, where 80 is a threshold level) to a clinical department instead of the claim examiner.

In another non-limiting example, there is a medication authorization request with a degrees of caution score of 70 for a patient in a jurisdiction state of Texas, where the NCPDP requires scores below a degrees of caution score of 75 to receive prior authorization from a clinical group. As 75 is a threshold level, the medication authorization request with the degrees of caution score of 70 is routed to the clinical group.

In some embodiments, the routing and decision module may utilize an escalation process to notify an examiner of a pending request. For example, the routing and decision module may send a notification (GUI notification, email, etc.) to the examiner regarding the pending request. In some embodiments, the examiner is required to respond within a defined time period or take action to obtain more information (or assistance). In some embodiments, the degrees of caution score is used as a component to route or escalate the medication authorization request.

In some embodiments, when the routing and decision module automatically approves a medication authorization request, the routing and decision module automatically transmits the approval to a pharmacy for the approved medication prescription to be filled at the pharmacy.

In some embodiments, machine learning is used to automate the routing and/or decision making process described herein. For example, in some embodiments, a machine learning module uses Azure AI + Machine Learning resources to develop predictive outcomes and claim trends reflected in the scoring model. For example, in some embodiments, the machine learning module may utilize patient data and National Council on Compensation Insurance (NCCI) Nature of Injury and Body Part codes to determine severity of particular injury types for patients. The machine learning module uses predictive modeling to determine a severity of an injury or an impact of a medication therapy as it relates to cost, medical appropriateness, and proper use. By providing key data points (i.e. NCCI Nature of Injury, NCCI Body Part, Patient Age and Gender), machine learning can correlate the output to lengthier claims with a greater span of drug usage (e.g., pain medications for treatment, drugs for digestive help, medications for sleep, etc.). For example, a 70-year-old female with a broken hip is more likely to end up with a longer, more costly claim due to the likelihood of a long recovery offset with other medications. While this example points out an obvious scenario, Machine Learning can identify less obvious patterns that result in lengthier claims.

In some embodiments, the degrees of caution score is presented to a user via a user interface. In some embodiments, the rules engine provides a degrees of caution score prior to the medication being dispensed. In such an embodiment, an individual, such as a claim manager, may review the degrees of caution score before making a decision to authorize or deny dispensing the medication. In some embodiments, the user interface presents the degrees of caution score using a temperature visualization. For example, in some embodiments, the degrees of caution score depicts low scores associated with green (for example, green text or green shading) and higher scores associated with red (for example, red text or red shading). A user can use the degrees of caution score as a guide, much in the same way a credit score is a factor in making the decision around approving a loan. High degrees of caution scores indicate more risk associated with approving the medication and therefore may prompt the examiner to, for example, review medication history, request a letter of medical necessity, contact the clinical team for assistance, and/or take some other action before approving the medication authorization request.

In some embodiments, automatic approval (e.g., due to an extremely low score) or denial (e.g., due to an extremely high score) is performed. In alternative embodiments, the user interface is configured to present the information to the claims examiner to make the decision. In some embodiments, an client (e.g., an insurance carrier) may instruct automatic approval and/or automatic denial based on a specific degrees of caution score. As an example, the client may define automatic approval for a medication request where the degrees of caution score is less than 15 (meaning a low risk medication). In this case, the system will not create a medication request and assign to an Examiner for review, but rather the system will automatically create the prior authorization and notify the pharmacy to reprocess.

In some embodiments, the automatic approval or automatic denial is based on one or more of the components of the degrees of caution score (e.g., a category score).

In some embodiments, the rules engine and/or routing and decision module are written in SQL and C# code. The variables, weights, and/or scores are calculated and/or stored in a SQL database, which allows for users to review scoring over time and adjust weights accordingly.

Although the described systems, methods, and/or non-transitory computer-readable medium are described in relation to workers’ compensation claims’ management practice, the described systems, methods, and/or non-transitory computer-readable medium have similar application benefits along the entire health care management spectrum.

The described systems and methods streamline the claims’ management process and minimize insurance abuse. The described systems and methods enhance the claims management process by automatically approving or denying medication authorization requests or providing the claims examiner with consumable data points that help them make decisions to approve or deny a medication authorization request in less time. The described systems and methods further minimize “rubber stamp” approvals by automating the process or putting the decision in the right hands. The “rubber stamp”’ approvals is a key problem in the industry as examiners are often burdened with many other tasks and managing patient’s medication therapy may or may not be a skill they possess. Thus, the described systems and methods assist in ensuring that safe decisions are made in order to improve the lives of patients by making prospective and concurrent clinical oversight a priority.

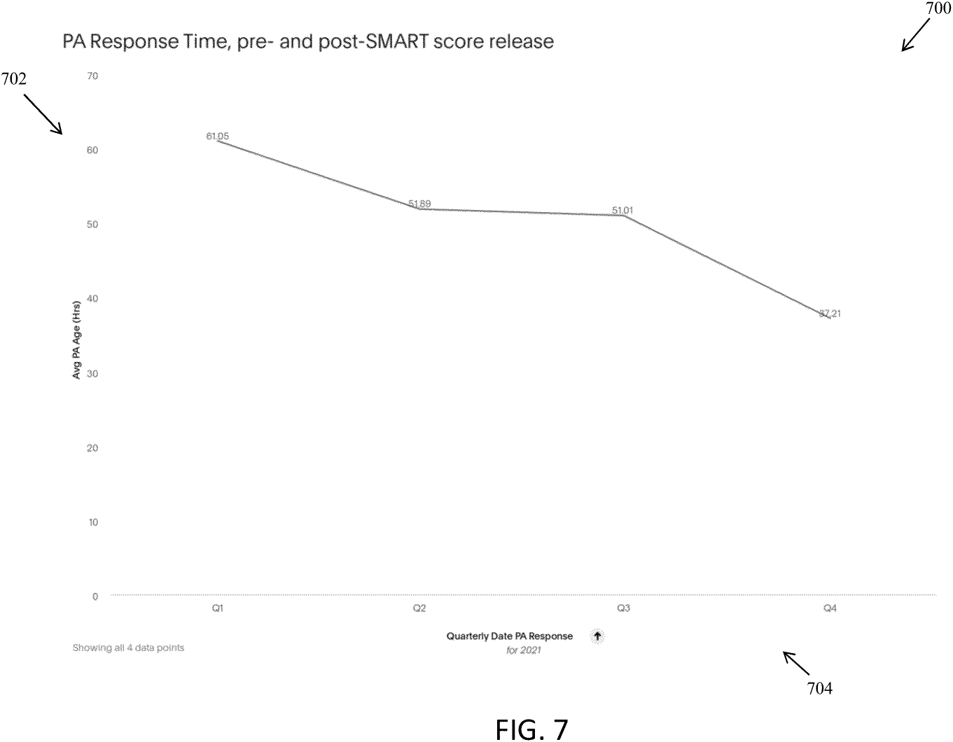

In addition, the described systems and methods improve authorization response times, thereby reducing the amount of time it takes for a prescription to be routed, decisioned, and completed. An analysis of prior authorization responses over an extended period (roughly 12 weeks) indicates a decrease in the overall response time when the degrees of caution score is presented. There is a demonstrative reduction in turn-around time for prior authorizations, as described in FIG. 7 below.

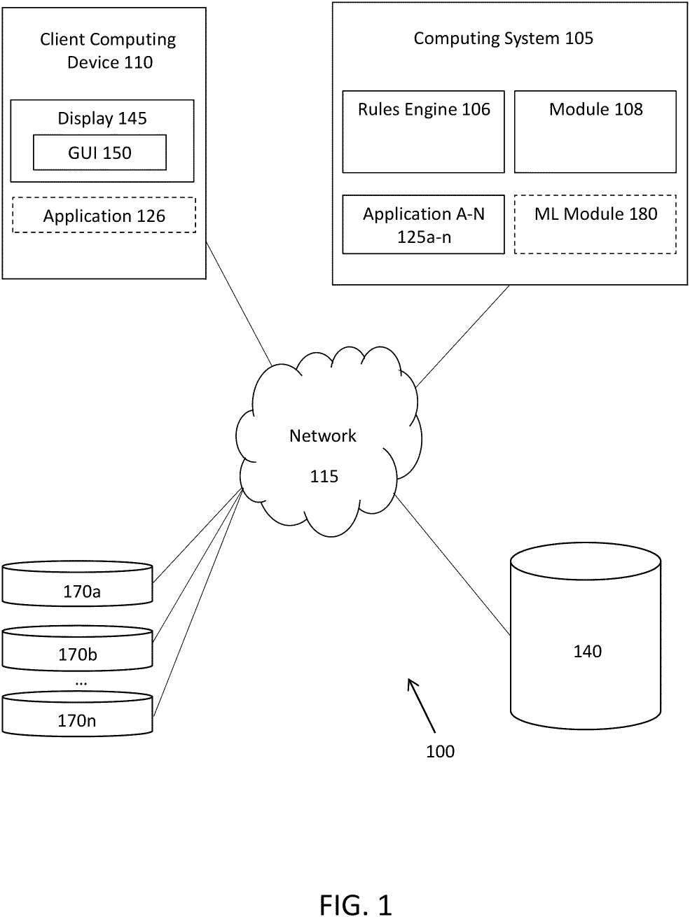

FIG. 1 schematically depicts a network diagram 100 of computing systems, devices, networks and databases that could be employed in connection with some embodiments described herein. The depicted network diagram 100 shows a computing system 105 communicating with a client computing device 110, databases 140, and data repositories 170a-n over network 115. The computing system 105 can host and execute a rules engine 106, a routing and decision module 108, and applications A-N 127a-n. It can be appreciated that each of the rules engine 106, routing and decision module 108, and applications A-N 125a-n can be executed on the same or separate computing systems. As described herein, the rules engine 106 and the routing and decision module 108 uses one or more of transaction driven variables, patient profile characteristics, medication therapy, prescribing patterns, clinical expertise, and machine learning to automate the routing and/or decision making process. The computing system 105 can further communicate with disparate data repositories 170a-n over the network 115 to retrieve data.

The computing system 105 can host one or more applications A-N 125a-n configured to interact with one or more components and/or facilitate access to the content of the databases 140 and data repositories 170a-n. The databases 140 and data repositories 170a-n may store information/data as described herein. For example, the databases 140 can include, but is not limited to, degrees of caution scores output from the rules engine 106, category scores output from the rules engine 106, studies, patient profile variables, therapy patterns variables, prescribing patterns variables, medication specifications variables, and prescriber and pharmacy quality variables. The databases 140 can be located at one or more geographically distributed locations from the computing system 105. Alternatively, the databases 140 can be located at the same geographic location as the computing system 105.

The data repositories 170a-n can store patient information and medical information stored by third parties, such as pharmacies, claim managers, clinicians, health care provider systems, payers (e.g., insurance companies), and medical professionals. For example, a user may store patient demographics and patient profile data; medication data may be stored and provided by a prescribing pharmacy; fill history may be stored and provided by businesses; studies may be stored and provided by businesses; and pharmacy and prescriber quality scores may be stored and provided by business and/or users.

The computing system 105 can be a physical computing device, a virtual computing device, or multiple physical and virtual computing devices functioning as one computing device. In some embodiments, the computing device 105 can be a cloud-based system providing computational functionality remote from the facility where the system is deployed. In some embodiments, the computing device 105 can operate on a software stack including an operating system, including but not limited to Microsoft Windows-based distributions and *nix-based distributions.

A graphical user interface 150 can be rendered on the display 145 of the client computing device 110. The term “UI” refers to a user interface, which is the point of human-computer interaction and communication in a device or system. This can include display screens, keyboards, a mouse and the appearance of a desktop. A user interface can also refer to a way through which a user interacts with an application or a website. For example, an application or a website can be configured to output information, such as the degrees of caution scores, to be rendered on the graphical user interface 150 rendered on the display 145.

The computing system 105 may generate and/or serve content such as web pages, for example, to be displayed by a browser of client computing device 110 over network 115 such as the Internet. In some embodiments, an application 126 is executed by the client computing device 110 and is accessed by a user of the client computing device 110. In some embodiments, application 126 is a software application, such as a mobile “app”, that can be downloaded to the client computing device 110 from the computing system 105. In some embodiments, application 126 provides a graphical user interface (GUI) 150 for enabling the functionality described herein, when executed on the client computing device 110.

A computing device embodied fully or in part as computing system 105 and/or client computing device 110 may be capable of sending or receiving signals, such as via a wired or wireless network, or may be capable of processing or storing signals, such as in memory as physical memory states. Devices and systems capable of operating as computing system 105 include, but are not limited to, as examples, one or more of dedicated rack-mounted servers, desktop computers, laptop computers, set top boxes, integrated devices combining various features, such as two or more features of the foregoing devices, or the like. Embodiments of computing system 105 may vary widely in configuration or capabilities, but generally may include one or more central processing units and memory. Computing system 105 may also include one or more mass storage devices, one or more power supplies, one or more wired or wireless network interfaces, one or more input/output interfaces, or one or more operating systems, such as Windows® Server, Mac® OS X®, Unix®, Linux®, FreeBSD®, or the like. Computing system 105 may include multiple different computing devices. Computing system 105 may include multiple computing devices that are networked with each other. Computing system 105 may include networks of processors or may employ networks of remote processors for processing (e.g., cloud computing). Some aspects may be implemented, at least in part, via a cloud container engine.

The computing system 105 may include a device that includes a configuration to provide content via a network to another device. The computing system 105 may further provide a variety of services that include, but are not limited to, web services, third-party services, audio services, video services, email services, instant messaging (IM) services, SMS services, MMS services, FTP services, voice over IP (VOIP) services, calendaring services, photo services, or the like. Examples of content may include text, images, audio, video, or the like, which may be processed in the form of physical signals, such as electrical signals, for example, or may be stored in memory, as physical states, for example. Examples of devices that may operate as or be included in computing system 105 include desktop computers, multiprocessor systems, microprocessor-type or programmable consumer electronics, etc.

A network may couple devices so that communications may be exchanged, such as between a server and a client device or other types of devices, including between wireless devices coupled via a wireless network, for example. A network may also include mass storage, such as network attached storage (NAS), a storage area network (SAN), or other forms of computer or machine readable media, for example. A network may include the Internet, one or more local area networks (LANs), one or more wide area networks (WANs), wire-line type connections, wireless type connections, or any combination thereof. Likewise, sub-networks, such as may employ differing architectures or may be compliant or compatible with differing protocols, may interoperate within a larger network. Various types of devices may, for example, be made available to provide an interoperable capability for differing architectures or protocols. As one illustrative example, a router may provide a link between otherwise separate and independent LANs.

A communication link or channel may include, for example, analog telephone lines, such as a twisted wire pair, a coaxial cable, full or fractional digital lines including T1, T2, T3, or T4 type lines, Integrated Services Digital Networks (ISDNs), Digital Subscriber Lines (DSLs), wireless links including satellite links, or other communication links or channels, such as may be known to those skilled in the art. Furthermore, a computing device or other related electronic devices may be remotely coupled to a network, such as via a telephone line or link, for example.

A wireless network may couple client devices with a network 115. A wireless network 115 may employ stand-alone ad-hoc networks, mesh networks, Wireless LAN (WLAN) networks, cellular networks, or the like. A wireless network 115 may further include a system of terminals, gateways, routers, or the like coupled by wireless radio links, or the like, which may move freely, randomly or organize themselves arbitrarily, such that network topology may change, at times even rapidly. A wireless network 115 may further employ a plurality of network access technologies, including Long Term Evolution (LTE), WLAN, Wireless Router (WR) mesh, or 2nd, 3rd, 4th, 5th, or 6th generation (2G, 3G, 4G, 5G, 6G) cellular technology, or the like. Network access technologies may enable wide area coverage for devices, such as client devices with varying degrees of mobility, for example.

For example, a network 115 may enable RF or wireless type communication via one or more network access technologies, such as Global System for Mobile communication (GSM), Universal Mobile Telecommunications System (UMTS), General Packet Radio Services (GPRS), Enhanced Data GSM Environment (EDGE), 3GPP Long Term Evolution (LTE), LTE Advanced, Wideband Code Division Multiple Access (WCDMA), Bluetooth, 802.11b/g/n, or the like. A wireless network may include virtually any type of wireless communication mechanism by which signals may be communicated between devices, such as a client device or a computing device, between or within a network, or the like.

In one embodiment, the client computing device 110 is a smartphone. In another embodiment, the client computing device 110 is a tablet. The client computing device 110 can also be a computer, a set-top box, a smart TV, or any other computing device.

In one embodiment, the rules engine 106, the routing and decision module 108, and/or the applications A-N 125a-n may be implemented in a “cloud computing” environment or as a “software as a service” (SaaS). For example, at least some of the operations may be performed by a group of computers (as examples of machines including processors), with these operations being accessible via a network (e.g., the Internet) and via one or more appropriate interfaces (e.g., APIs). Example embodiments may be implemented in digital electronic circuitry, or in computer hardware, firmware, software, or in combinations of them. Example embodiments may be implemented using a computer program product, for example, a computer program tangibly embodied in an information carrier, for example, in a machine-readable medium for execution by, or to control the operation of, data processing apparatus, for example, a programmable processor, a computer, or multiple computers.

In one embodiment, the client computing device 110a can be operated by a user. In some embodiments, users may be claim managers, clinicians, health care provider systems, payers (e.g., insurance companies), and medical professionals. In some embodiments, a user can execute application 126 on the client computing device 110 to interface with the computing system 105. In some embodiments, an application 126 can render a GUI 150 on the display 145. It can be appreciated, that, in some embodiments, the GUI 150 can be different for each type of user.

In some embodiment, the rules engine 106 and/or routing and decision module 108 can access data from the data repositories 170a-n. The data can include, but is not limited to, identification information associated with the patient, prescription history, health care providers, information associated with a patient’s illness, information associated with a patient’s medical condition, and/or information associated with the patient’s treatment. In some embodiments, data is stored in SQL database(s). In addition, in some embodiments, scores and weights are stored in SQL database(s), which allows for users to review scoring over time and adjust the weights accordingly.

In some embodiments, the rules engine 106 and/or routing and decision module 108 is written in a combination of SQL and C# code.

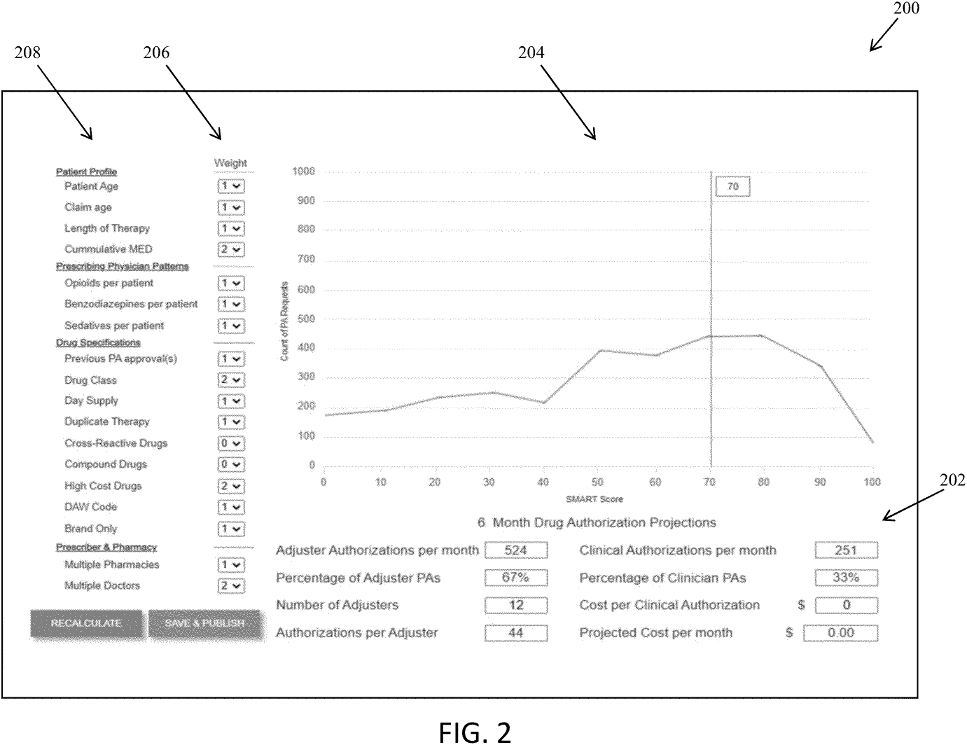

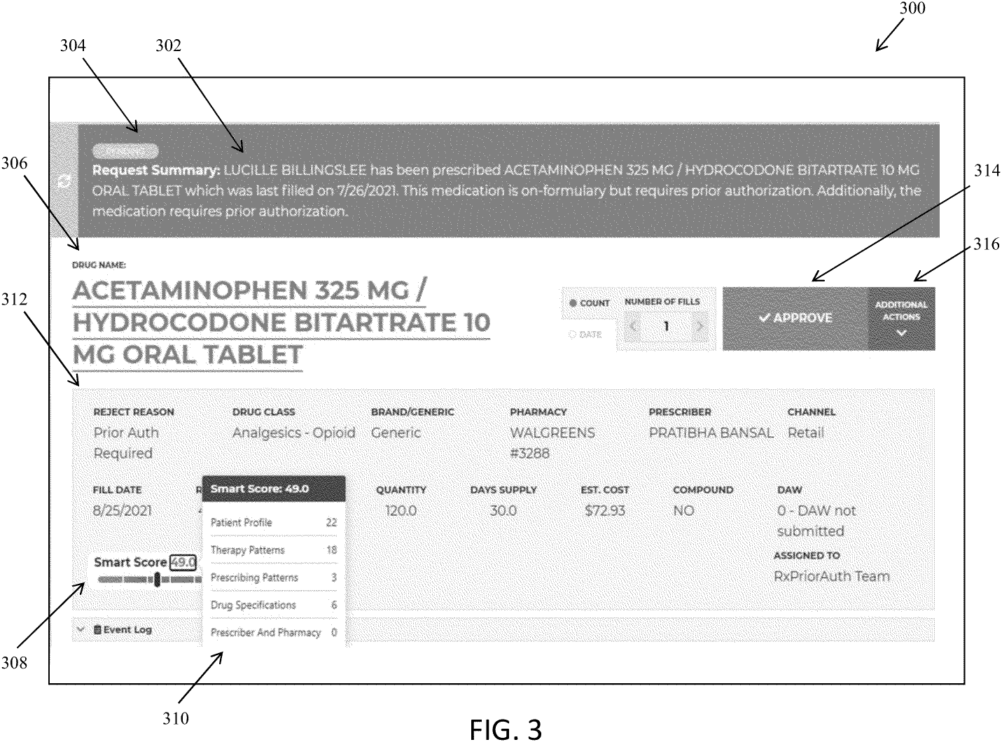

In some embodiments, the graphical user interfaces illustrated in FIG. 2 and/or FIG. 3 are is presented to a user via the GUI 150. In some embodiments, a graphical user interface 150 of the client computing device 110 is used to accept input from a user, such as variable weights, approvals or denials of medication authorization requests, and/or threshold levels.

In some embodiments, computing system 105 includes a machine learning module 180 to automate the routing and/or decision making process. For example, in some embodiments, the machine learning module 180 uses Azure AI + Machine Learning resources to develop predictive outcomes and claim trends reflected in the scoring model.

FIG. 2 illustrates an exemplary graphical user interface (GUI) 200 for customizing weights assigned to variables and viewing authorization projections based on the assigned weights according to the present disclosure. GUI 200 provides a modeling tool that allows a user to view the degrees of caution score, analyze authorization history, and develop a desired model for adjuster versus clinical routing. GUI 200 provides medication authorization projections 202 based on the degrees of caution score. The medication authorization projections 202 can include, but are not limited to, a number of adjuster authorizations per month, a number of clinical authorizations per month, a percentage of adjuster prior authorizations, a percentage of clinician prior authorizations, a number of adjusters, a number of authorizations per adjuster, a cost per clinical authorization, and projected cost per month.

In the illustrated example, the GUI 200 displays an aggregate view 204 of degrees of caution scores based on 6 months of claim history. Using a sidebar, the user can adjust weights 206 for variables 208 to define an importance of particular variables. Adjusting weights for one or more variables may modify the degrees of caution score. The user can review scoring over time and adjust the weights accordingly.

In some embodiments, each variable has a default weighting that is used to increase or decrease the impact of that variable on the degrees of caution score. A user can customize this weighting to increase or decrease the impact of the variable. In some embodiments, the default weighting can range from 0 to 5.

In some embodiments, the rules engine automatically scores each variable on a scale from 0 to 4 that defines the risk associated with approving the medication based on the variable. In some embodiments, the user manually scores each variable on a scale from 0 to 4 that defines the risk associated with approving the medication. The rules engine then multiplies the assigned score by the default weighing or the weighting assigned by the user.

Each of these variables can be included or excluded and/or weighted by a user when calculating the degrees of caution score.

FIG. 3 illustrates an exemplary graphical user interface (GUI) 300 for reviewing and approving or denying a medication authorization request according to the present disclosure.

In some embodiments, a user, such as a claims manager, uses the GUI 300 for reviewing and decisioning a medication authorization request. In some embodiments, the GUI 300 presents a request summary 302, a request status 304 (for example, pending), and a medication name 306. The GUI 300 further presents a degrees of caution score 308 and categories with associated category scores 310, in addition to several other variables 312 that may be used in making the decision. The variables 312 may include, but are not limited to, a rejection reason, a fill date, a medication class, whether the medication is a brand name or a generic, a quantity of medication, a name of the filling pharmacy, a days supply, an estimated cost for the medication, a name of the prescriber, whether the medication is a compound medication, a dispensing channel, whether the medication authorization request includes a Dispense as Written (DAW) code or instructions, and an assignment of the medication authorization request.

In some embodiments, the user can approve the medication and/or authorized dispensing the medication to the patient by selecting an approval button 314 in the GUI 300.

In some embodiments, the user can take additional actions other than approving the medication authorization request by selecting an additional actions button 316 in the GUI 300, whereby additional actions are presented to the user in a drop down menu. For example, the user can deny the medication authorization request, requests a letter of medical necessity, request a utilization review by a third party before decisioning, or reassign to a clinician for a determination.

In some embodiments, automatic approval and deny rules determine whether the medication authorization request is approved or denied. In some embodiments, the user can still review information regarding the medication authorization request using the GUI 300. In some embodiments, the user makes the final determination regarding approving or denying the medication authorization request using the GUI 300.

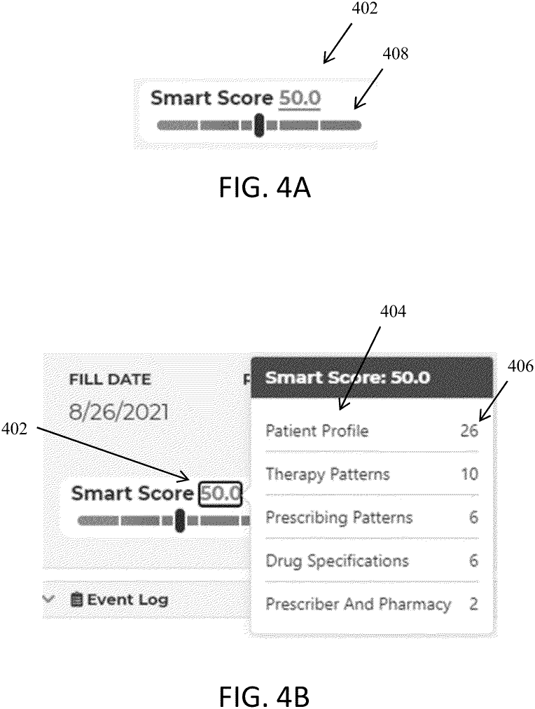

As described above, the rules engine uses a combination of variables to determine the degrees of caution score 308. In the example, the degrees of caution score 308 for the medication 306 is 49.0. In the example, the degrees of caution score is broken into five categories 310, Patient Profile, Therapy Patterns, Prescribing Patterns, Medication Specifications, and Prescriber & Pharmacy Quality, where each category includes a score. The degrees of caution score and category scores are further described in FIGS. 4A and 4B.

FIGS. 4A and 4B illustrate the exemplary degrees of caution score 402 broken into categories 404 with associated scores 406 as shown on a graphical user interface (GUI) in accordance with the present disclosure.

In some embodiments, a score for a variable is multiplied by a default weighting from 0 to 5 to generate a weighted score. After each variable is assigned a weighted score, the rules engine sums the weighted scores of the variables for each category 404, giving each category a score 406, as shown in FIG. 4B. In the example, the degrees of caution score 402 is broken into five categories 404, Patient Profile, Therapy Patterns, Prescribing Patterns, Medication Specifications, and Prescriber & Pharmacy Quality, where each category 404 includes a score 406.

The Patient Profile category incorporate variables that are pertinent to the patient, such as the patient age (50), multiple claims (no), age of the claim (~8 years), injury type (sprain/strain), injury location (back), and jurisdiction state of the claim (Longshore which typically means fishing, oil or offshore work). In the example, the Patient Profile category received a score of 26.

The Therapy Patterns category incorporates the medication history for this patient (58 transactions), channel source (Retail Pharmacy), cross reactive medications, and the patient MED (Morphine equivalent dose) level (22). In the example, the Therapy Patterns category received a score of 10.

The Prescribing Patterns category incorporates variables that measure behaviors of the prescribing doctor. For example, this is used to identify doctors who are over prescribing various Class II medications (i.e. opioids, benzodiazepines, sleep agents and muscle relaxers) and combinations of these. In the example, the Prescribing Patterns category received a score of 6.

The Medication Specifications category incorporates variables includes specifics of the requested medication. Examples include the medication class, quantity, day supply, compound, repackaged, topical, Dispense As Written (DAW), previous approval and atypical workers’ compensation medications. In the example, the Medication Specifications category received a score of 6.

The Prescriber & Pharmacy Quality category incorporates the potential use of multiple doctors or pharmacies by the patient (commonly used to hide medications or duplicate prescription usage), quality of the doctor and pharmacy network affiliation (for example, catching compounding pharmacies, mail order facilities or less common distribution facilities). In the example, the Prescriber & Pharmacy Quality category received a score of 2.

The rules engine sums all category scores 406 to obtain the degrees of caution score 402. In the example, the degrees of caution score 402 for the medication is 50.

Examples of additional variables not used in the degrees of caution score 402, may include, but is not limited to, industry code/SIC code, smoker, marijuana use, medication testing results or return to work status. In some embodiments, these variables would be provided by the client.

In the example, the degrees of caution score 402 is associated with a temperature visualization bar 408 depicting where the degrees of caution score 402 falls within a temperature spectrum, with low scores associated with green (less risk or “go”) and higher scores associated with red (more risk or “stop”).

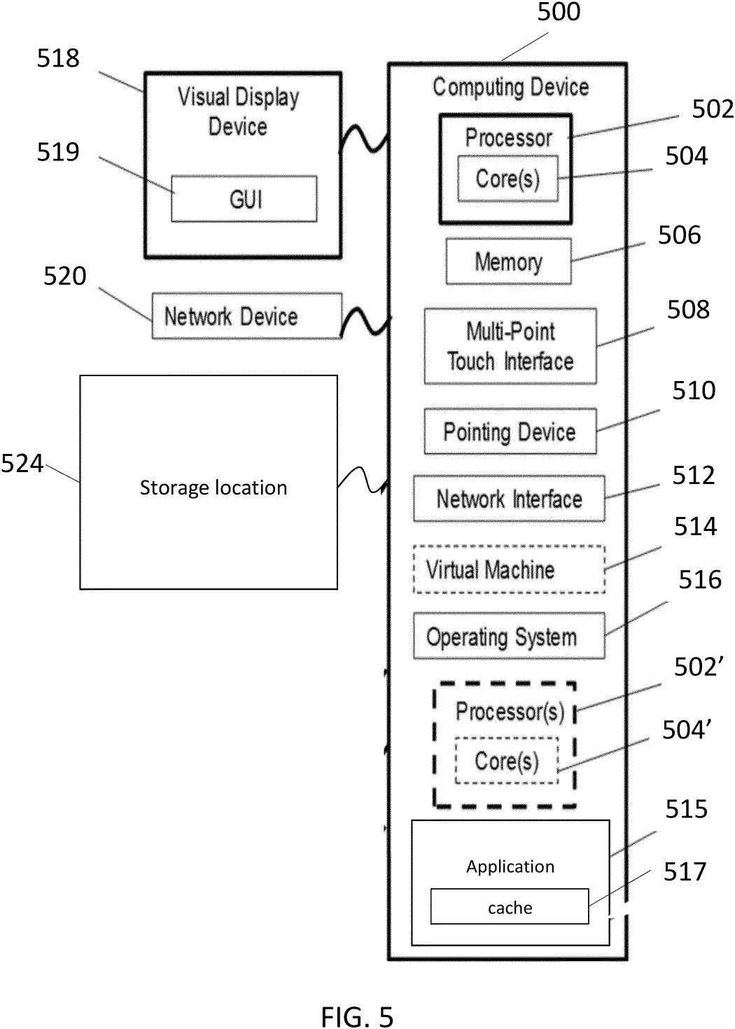

FIG. 5 is a block diagram of an example computing device 500 that can be used to perform one or more steps of the systems and methods provided by exemplary embodiments. In an exemplary embodiment, computing device 500 is computing device 105 and/or client computing device 110 illustrated in FIG. 1. Computing device 500 includes one or more processors 502. Computing device 500 includes one or more non-transitory computer-readable media for storing one or more computer-executable instructions or software for implementing exemplary embodiments such as the prioritization module described herein. The non-transitory computer-readable media can include, but are not limited to, one or more types of hardware memory, non-transitory tangible media (for example, one or more magnetic storage disks, one or more optical disks, one or more USB flash drives), and the like. For example, a memory 506 included in computing device 500 can store computer-readable and computer-executable instructions or software for implementing exemplary embodiments. Computing device 500 also includes a core 504 associated with processor 502, and optionally, one or more additional processor(s) 502′ and associated core(s) 504′ (for example, in the case of computer systems having multiple processors/cores), for executing computer-readable and computer-executable instructions or software stored in memory 506 and other programs for controlling system hardware. Processor 502 and processor(s) 502′ can each be a single core processor or multiple core (504 and 504′) processor. Computing device 500 may include an browser application 515 and a browser cache 517. As described above, the browser application 515 can enable a user to view graphical user interfaces.

Virtualization can be employed in computing device 500 so that infrastructure and resources in the computing device can be shared dynamically. A virtual machine 514 can be provided to handle a process running on multiple processors so that the process appears to be using only one computing resource rather than multiple computing resources. Multiple virtual machines can also be used with one processor.

Memory 506 can include a computer system memory or random access memory, such as DRAM, SRAM, EDO RAM, and the like. Memory 506 can include other types of memory as well, or combinations thereof. In some embodiments, a user can interact with computing device 500 through a visual display device 518, such as a touch screen display or computer monitor, which can display one or more user interfaces. Visual display device 518 may also display other aspects, elements and/or information or data associated with exemplary embodiments. Computing device 500 may include other I/O devices for receiving input from a user, for example, a keyboard or any suitable multi-point touch interface 508, a pointing device 510 (e.g., a pen, stylus, mouse, or trackpad). The keyboard 508 and pointing device 510 may be coupled to visual display device 518. Computing device 500 may include other suitable conventional I/O peripherals.

Computing device 500 can also include one or more storage devices 524, such as a hard-drive, CD-ROM, or other computer readable media, for storing data and computer-readable instructions and/or software that implements embodiments of the system, as described herein, or portions thereof. Exemplary storage device 524 can also store one or more storage devices for storing any suitable information required to implement exemplary embodiments. For example, storage device 524 and/or memory 506 store portions of data and/or files.

Computing device 500 can include a network interface 512 configured to interface via one or more network devices 522 with one or more networks, for example, Local Area Network (LAN), Wide Area Network (WAN) or the Internet through a variety of connections including, but not limited to, standard telephone lines, LAN or WAN links (for example, 802.11, T1, T3, 56kb, X.25), broadband connections (for example, ISDN, Frame Relay, ATM), wireless connections, controller area network (CAN), or some combination of any or all of the above. The network interface 512 can include a built-in network adapter, network interface card, PCMCIA network card, card bus network adapter, wireless network adapter, USB network adapter, modem or any other device suitable for interfacing computing device 500 to any type of network capable of communication and performing the operations described herein. Moreover, computing device 500 can be any computer system, such as a workstation, desktop computer, storage server, laptop, handheld computer, tablet computer (e.g., the iPad® tablet computer), mobile computing or communication device (e.g., the iPhone® communication device), or other form of computing or telecommunications device that is capable of communication and that has sufficient processor power and memory capacity to perform the operations described herein.

Computing device 500 can run any operating system 516, such as any of the versions of the Microsoft® Windows® operating systems, the different releases of the Unix and Linux operating systems, any version of the MacOS® for Macintosh computers, any embedded operating system, any real-time operating system, any open source operating system, any proprietary operating system, any operating systems for mobile computing devices, or any other operating system capable of running on the computing device and performing the operations described herein. In exemplary embodiments, the operating system 516 can be run in native mode or emulated mode. In an exemplary embodiment, the operating system 516 can be run on one or more cloud machine instances.

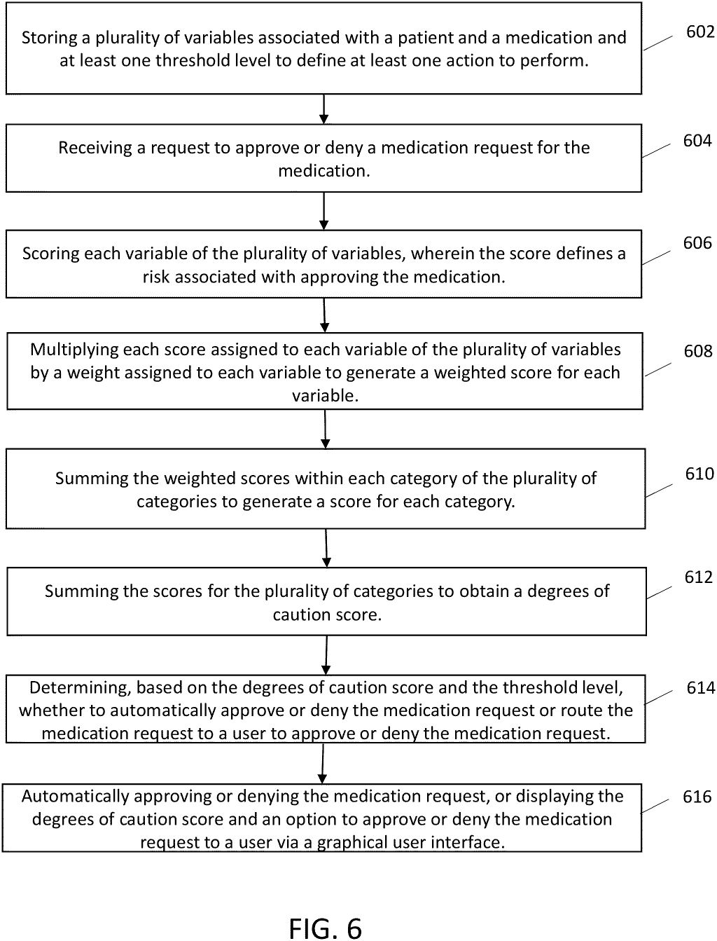

FIG. 6 is a flow chart of an example process that can be executed in accordance with some embodiments of the present disclosure. Step 602 includes storing, within a storage, a plurality of variables associated with a patient and a medication, wherein each variable of the plurality of variables are associated with a category of a plurality of categories, and at least one threshold level to define at least one action to perform. Step 604 includes receiving, via one or more processors in communication with the storage, a request to approve or deny a medication authorization request for the medication. Step 606 includes scoring, via the one or more processors, each variable of the plurality of variables, wherein the score defines a risk associated with approving the medication. Step 608 includes multiplying, via the one or more processors, each score assigned to each variable of the plurality of variables by a weight assigned to each variable to generate a weighted score for each variable. Step 610 includes summing, via the one or more processors, the weighted scores within each category of the plurality of categories to generate a score for each category. Step 612 includes summing, via the one or more processors, the scores for the plurality of categories to obtain a degrees of caution score. Step 614 includes determining, via the one or more processors, based on the degrees of caution score and the threshold level, whether to automatically approve or deny the medication authorization request or route the medication authorization request to a user to approve or deny the medication authorization request. Step 616 includes automatically approving or denying, via the one or more processors, the medication authorization request, or displaying the degrees of caution score and an option to approve or deny the medication authorization request to a user via a graphical user interface.

FIG. 7 is a chart demonstrating a reduction in turn-around time for prior authorizations using degrees of caution scores. In the example, degrees of caution scores began to be utilized in the first quarter (Q1). The y-axis 702 of chart 700 represents the average prior authorization age (in hours). The x-axis 704 of chart 700 represents the quarterly dates. As shown, the described systems and methods improve authorization response times over the four quarters, thereby reducing the amount of time it takes for a prescription to be routed, decisioned, and completed. An analysis of prior authorization responses over an extended period (roughly 12 weeks) indicates a decrease in the overall response time when the degrees of caution score is presented.

The description herein is presented to enable any person skilled in the art to create and use a computer system configuration and related method and systems for managed authorization routing. Various modifications to the example embodiments will be readily apparent to those skilled in the art, and the generic principles defined herein may be applied to other embodiments and applications without departing from the spirit and scope of the present disclosure. Moreover, in the following description, numerous details are set forth for the purpose of explanation. However, one of ordinary skill in the art will realize that embodiments of the present disclosure may be practiced without the use of these specific details. In other instances, well-known structures and processes are shown in block diagram form in order not to obscure the description of embodiments of the present disclosure with unnecessary detail. Thus, the present disclosure is not intended to be limited to the embodiments shown, but is to be accorded the widest scope consistent with the principles and features disclosed herein.

In describing exemplary embodiments, specific terminology is used for the sake of clarity. For purposes of description, each specific term is intended to at least include all technical and functional equivalents that operate in a similar manner to accomplish a similar purpose. Additionally, in some instances where a particular exemplary embodiment includes a plurality of system elements, device components or method steps, those elements, components or steps can be replaced with a single element, component, or step. Likewise, a single element, component, or step can be replaced with a plurality of elements, components, or steps that serve the same purpose. Moreover, while exemplary embodiments have been shown and described with references to particular embodiments thereof, those of ordinary skill in the art will understand that various substitutions and alterations in form and detail can be made therein without departing from the scope of the present disclosure. Further still, other aspects, functions, and advantages are also within the scope of the present disclosure.

Exemplary flowcharts are provided herein for illustrative purposes and are non-limiting examples of methods. One of ordinary skill in the art will recognize that exemplary methods can include more or fewer steps than those illustrated in the exemplary flowcharts, and that the steps in the exemplary flowcharts can be performed in a different order than the order shown in the illustrative flowcharts.

Portions or all of the embodiments may be provided as one or more computer-readable programs or code embodied on or in one or more non-transitory mediums. The mediums may be, but are not limited to a hard disk, a compact disc, a digital versatile disc, a flash memory, a PROM, a RAM, a ROM, or a magnetic tape. In general, the computer-readable programs or code may be implemented in many computing languages.

1. A system for managed authorization routing, the system comprising:
storage configured to hold:
a plurality of variables associated with a patient and a medication, wherein each variable of the plurality of variables are associated with a category of a plurality of categories, and
at least one threshold level to define at least one action to perform; and
one or more processors in communication with the storage and configured to execute instructions comprising instructions to:
receive a request to approve or disapprove a medication authorization request for the medication;
score each variable of the plurality of variables, wherein the score defines a risk associated with approving the medication;
multiply each score assigned to each variable of the plurality of variables by a weight assigned to each variable to generate a weighted score for each variable;
sum the weighted scores within each category of the plurality of categories to generate a score for each category;
sum the scores for the plurality of categories to obtain a degrees of caution score;
determine, based on the degrees of caution score and the threshold level, whether to automatically approve or deny the medication authorization request or route the medication authorization request to a user to approve or deny the medication authorization request; and
automatically approve or deny the medication authorization request, or display the degrees of caution score and an option to approve or deny the medication authorization request to a user via a graphical user interface.

2. The system of claim 1, wherein the degrees of caution score ranges from 1 and 100, wherein a higher degrees of caution score indicates risk for complications, dependency, or improper use associated with approving the medication authorization request.

3. The system of claim 1, wherein the plurality of categories include a patient profile category, therapy patterns category, prescribing patterns category, medication specifications category, and prescriber & pharmacy quality category.

4. The system of claim 1, wherein one or more of the variables of the plurality of variables include patient profile metrics, clinical expertise, prescribing patterns of the physician and previous disposition, claim age, length of therapy, medication class, medication quantity, day supply, duplicate therapies, cross-reactive medications, multiple pharmacies, multiple prescribers, transaction source, physician quality scores, total and cumulative opioid dosage, medication testing, medication monitoring, use of compounds, use of repackaged medications, or brand only preference.

5. The system of claim 1, wherein the at least one threshold level is defined by a user to determine a particular action.

6. The system of claim 1, the one or more processors configured to execute instructions comprising instructions to utilize machine learning to automate the approval or denial of the medication authorization request.

7. The system of claim 1, wherein the at least one actions associated with the at least one threshold level includes one or more of all medication authorization requests with degrees of caution scores below a first threshold level are automatically approved, all medication authorization requests with degrees of caution scores above the first threshold level and below a second threshold level are transmitted for manual review, and all medication authorization requests with degrees of caution scores above the second threshold level are automatically denied.

8. A method for managed authorization routing, the method comprising:
storing, within a storage, a plurality of variables associated with a patient and a medication, wherein each variable of the plurality of variables are associated with a category of a plurality of categories, and at least one threshold level to define at least one action to perform;
receiving, via one or more processors in communication with the storage, a request to approve or deny a medication authorization request for the medication;
scoring, via the one or more processors, each variable of the plurality of variables, wherein the score defines a risk associated with approving the medication;
multiplying, via the one or more processors, each score assigned to each variable of the plurality of variables by a weight assigned to each variable to generate a weighted score for each variable;
summing, via the one or more processors, the weighted scores within each category of the plurality of categories to generate a score for each category;
summing, via the one or more processors, the scores for the plurality of categories to obtain a degrees of caution score;
determining, via the one or more processors, based on the degrees of caution score and the threshold level, whether to automatically approve or deny the medication authorization request or route the medication authorization request to a user to approve or deny the medication authorization request; and
automatically approving or denying, via the one or more processors, the medication authorization request, or displaying the degrees of caution score and an option to approve or deny the medication authorization request to a user via a graphical user interface.

9. The method of claim 7, wherein the degrees of caution score ranges from 1 and 100, wherein a higher degrees of caution score indicates risk for complications, dependency, or improper use associated with approving the medication authorization request.

10. The method of claim 7, wherein the plurality of categories include a patient profile category, therapy patterns category, prescribing patterns category, medication specifications category, and prescriber & pharmacy quality category.

11. The method of claim 7, wherein one or more of the variables of the plurality of variables include patient profile metrics, clinical expertise, prescribing patterns of the physician and previous disposition, claim age, length of therapy, medication class, medication quantity, day supply, duplicate therapies, cross-reactive medications, multiple pharmacies, multiple prescribers, transaction source, physician quality scores, total and cumulative opioid dosage, medication testing, medication monitoring, use of compounds, use of repackaged medications, or brand only preference.

12. The method of claim 7, wherein the at least one threshold level is defined by a user to determine a particular action.

13. The method of claim 7, further comprising utilizing machine learning to automate the approval or denial of the medication authorization request.

14. The method of claim 7, wherein the at least one actions associated with the at least one threshold level includes one or more of all medication authorization requests with degrees of caution scores below a first threshold level are automatically approved, all medication authorization requests with degrees of caution scores above the first threshold level and below a second threshold level are transmitted for manual review, and all medication authorization requests with degrees of caution scores above the second threshold level are automatically denied.

15. A non-transitory computer readable medium storing instructions executable by a processor, wherein execution of the instructions causes the processor to implement a method for identifying in an x-ray image, the method comprising:
storing, within a storage, a plurality of variables associated with a patient and a medication, wherein each variable of the plurality of variables are associated with a category of a plurality of categories, and at least one threshold level to define at least one action to perform;
receiving a request to approve or deny a medication authorization request for the medication;
scoring each variable of the plurality of variables, wherein the score defines a risk associated with approving the medication;
multiplying each score assigned to each variable of the plurality of variables by a weight assigned to each variable to generate a weighted score for each variable;
summing the weighted scores within each category of the plurality of categories to generate a score for each category;
summing the scores for the plurality of categories to obtain a degrees of caution score;
determining based on the degrees of caution score and the threshold level, whether to automatically approve or deny the medication authorization request or route the medication authorization request to a user to approve or deny the medication authorization request; and
automatically approving or denying the medication authorization request, or displaying the degrees of caution score and an option to approve or deny the medication authorization request to a user via a graphical user interface.

16. The non-transitory computer readable medium of 15, wherein the degrees of caution score ranges from 1 and 100, wherein a higher degrees of caution score indicates risk for complications, dependency, or improper use associated with approving the medication authorization request.

17. The non-transitory computer readable medium of 15, wherein one or more of the variables of the plurality of variables include patient profile metrics, clinical expertise, prescribing patterns of the physician and previous disposition, claim age, length of therapy, medication class, medication quantity, day supply, duplicate therapies, cross-reactive medications, multiple pharmacies, multiple prescribers, transaction source, physician quality scores, total and cumulative opioid dosage, medication testing, medication monitoring, use of compounds, use of repackaged medications, or brand only preference.

18. The non-transitory computer readable medium of 15, wherein the at least one threshold level is defined by a user to determine a particular action.

19. The non-transitory computer readable medium of 15, further comprising utilizing machine learning to automate the approval or denial of the medication authorization request.

20. The non-transitory computer readable medium of 15, wherein the at least one actions associated with the at least one threshold level includes one or more of all medication authorization requests with degrees of caution scores below a first threshold level are automatically approved, all medication authorization requests with degrees of caution scores above the first threshold level and below a second threshold level are transmitted for manual review, and all medication authorization requests with degrees of caution scores above the second threshold level are automatically denied.
